(19) United States
(12) Patent Application Publication
(10) Pub. No.: US 2019/0275049 A1
(43) Pub. Date: Sep. 12, 2019

(54) Combination of an EGFR T790M Inhibitor and a CDK Inhibitor for the Treatment of Non-Small Cell Lung Cancer

(71) Applicant: PFIZER INC., NEW YORK, NY (US)

(72) Inventors: Zelanna Iris GOLDBERG, San Diego, CA (US); Yuli WANG, San Diego, CA (US); Scott Lawrence WEINRICH, San Diego, CA (US); Keith David WILNER, La Jolla, CA (US)

(73) Assignee: PFIZER INC., NEW YORK, NY (US)

(21) Appl. No.: 16/349,941

(22) PCT Filed: Nov. 7, 2017
(86) PCT No.: PCT/IB2017/056952

§ 371 Date: May 14, 2019

Related U.S. Application Data
(60) Provisional application No. 62/423,146, filed on Nov. 16, 2016.
(60) Provisional application No. 62/571,114, filed on Oct. 11, 2017.

(51) Int. Cl. A61K 31/52; A61P 35/00

(52) U.S. Cl. CPC A61K 31/52; A61P 35/00

(57) ABSTRACT

This invention relates to a method of treating non-small cell lung cancer by administering an EGFR T790M inhibitor in combination with a CDK inhibitor to a patient in need thereof.

### CROSS-REFERENCE TO RELATED APPLICATIONS

This patent application claims the benefit of priority to U.S. Provisional Patent Application Ser. No. 62/423,146, filed Nov. 16, 2016, and U.S. Provisional Patent Application Ser. No. 62/571,114, filed Oct. 11, 2017, the contents of which are hereby incorporated by reference in their entireties.

### FIELD OF THE INVENTION

The present invention relates to combination therapies useful for the treatment of non-small cell lung cancer. In particular, this invention relates to methods for treating non-small cell lung cancer by administering an EGFR T790M inhibitor in combination with a CDK inhibitor. Pharmaceutical uses of the combination of the present invention are also described.

### BACKGROUND

Non-small cell lung cancer (NSCLC) is the leading cause of cancer death worldwide, with an estimated 1.4 million new cases diagnosed each year. In lung adenocarcinoma, which is the most common form of non-small cell lung cancer, patients harboring mutations in the epidermal growth factor receptor (EGFR) constitute between 10-30% of the overall population. It is in this segment of EGFR-mutant (EGFRm) NSCLC patients for whom EGFR tyrosine kinase inhibitors (TKIs) such as erlotinib or gefitinib can be most effective (Paez et al. Science 2004; Lynch et al. NEJM 2004; Pao et al. PNAS 2004). The most common activating mutations associated with good response to these inhibitors are deletions within exon 19 (e.g. delE746-A750, del 19) and point mutations in the activation loop (exon 21, in particular, L858R). Additional somatic mutations identified to date but to a lesser extent include point mutations: G719S, G719C, G719A, L861 and small insertions in Exon 20 (Shigematsu et a.l JNCI 2005; Fukuoka et al. JCO 2003; Kris et al. JAMA 2003; and Shepherd et al. NEJM 2004).

While EGFR TKIs, such as gefinitinb and erlotinib, can be effective first-line treatment for the EGFRm sub-population, the majority of patients who initially respond develop resistance. The primary mechanism of resistance, observed in approximately 60% of patients, is due to a second mutation (T790M) which occurs at the gatekeeper threonine residue (Kosaka et al CCR 2006; Balak et al CCR 2006 and Engelman et al Science 2007).

The compound, N-((3R,4R)-4-fluoro-1-(6-((3-methoxy-1-methyl-1H-pyrazol-4-yl)amino)-9-methyl-9H-purin-2-yl)pyrrolidin-3-yl)acrylamide or N-[(3R,4R)-4-fluoro-1-[6-[(3-methoxy-1-methyl-1H-pyrazol-4-yl)amino]-9-methyl-9H-purin-2-yl]-3-pyrrolidinyl]-2-propenamide (also referred to as “PF-06747775”), is a potent and selective EGFR T790M inhibitor, having the structure:

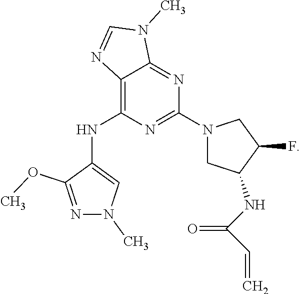 [SMILES: C=CC(=O)N[C@@H]1CN(c2nc(Nc3cn(C)nc3OC)c3ncn(C)c3n2)C[C@H]1F]

PF-06747775 and pharmaceutically acceptable salts thereof are disclosed in International Publication No. WO2015/075598 and U.S. Pat. No. 9290496. The contents of each of the foregoing references are incorporated herein by reference in their entirety.

PF-06747775 is being investigated in a Phase I/II clinical trial as a single agent in patients with advanced EGFR-mutant (del 19 or L858R, with or without T790M) NSCLC (ClinicalTrials.gov Identifier: NCT02349633).

Palbociclib is a potent and selective inhibitor of CDK4 and CDK6, having the structure:

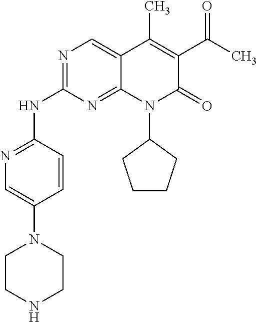 [SMILES: CC(=O)c1c(C)c2cnc(Nc3ccc(N4CCNCC4)cn3)nc2n(C2CCCC2)c1=O]

Palbociclib is described in WHO Drug Information, Vol. 27, No. 2, page 172 (2013). Palbociclib and pharmaceutically acceptable salts thereof are disclosed in International Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588. The contents of each of the foregoing references are incorporated herein by reference in their entirety.

Palbociclib is approved in the United States for the treatment of hormone receptor (HR)-positive, human epidermal growth factor 2 (HER2)-negative advanced or metastatic breast cancer in combination with letrozole as initial endocrine therapy or in combination with fulvestrant following disease progression on endocrine therapy. The recommended dose of palbociclib is 125 mg once daily for 21 consecutive days followed by 7 days off treatment to comprise a complete cycle of 28 days. The drug is sold by Pfizer under the trade name IBRANCE® in the form of an immediate release (IR) capsule dosage form comprising palbociclib as a free base for oral administration.

Improved therapies for the treatment of non-small cell lung cancer comprise a large unmet medical need and the identification of novel combination regimens are required to improve treatment outcome.

### SUMMARY OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein.

Embodiments described herein relate to a method for treating non-small cell lung cancer comprising administering to a patient in need thereof an amount of an EGFR T790M inhibitor and an amount of a CDK inhibitor, wherein the amounts together are effective in treating non-small cell lung cancer.

Additional embodiments described herein relate to a method for treating non-small cell lung cancer comprising administering to a patient in need thereof a synergistic amount of an EGFR T790M inhibitor in combination with a CDK inhibitor.

Further embodiments described herein relate to a combination of an EGFR T790M inhibitor and a CDK inhibitor for use in the treatment of non-small cell lung cancer.

Some embodiments described herein relate to a use of an EGFR T790M inhibitor and a CDK inhibitor, in the manufacture of a medicament for the treatment of non-small cell lung cancer.

Additional embodiments described herein relate to a combination of an EGFR T790M inhibitor and a CDK inhibitor for use in the treatment of non-small cell lung cancer, wherein the combination is synergistic.

Some embodiments described herein relate to a use of a synergistic amount of an EGFR T790M inhibitor and a CDK inhibitor, in the manufacture of a medicament for the treatment of non-small cell lung cancer.

In embodiments of the method or use of the present invention, the EGFR T790M inhibitor is irreversible.

In embodiments of the method or use of the present invention, the EGFR T790M inhibitor is selected from the group consisting of osimertinib, olmutinib, naquotinib, nazartinib, rociletinib, WZ4002 and TAS-2913, or a pharmaceutically acceptable salt thereof.

In embodiments of the method or use of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof.

In embodiments of the method or use of the present invention, the CDK inhibitor is a CDK 4/6 inhibitor.

In embodiments of the method or use of the present invention, the CDK 4/6 inhibitor is selected from the group consisting of abemaciclib, ribociclib and palbociclib, or a pharmaceutically acceptable salt thereof.

In embodiments of the method or use of the present invention, the CDK 4/6 inhibitor is palbociclib, or a pharmaceutically acceptable salt thereof.

In embodiments of the method or use of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and the CDK inhibitor is palbociclib, or a pharmaceutically acceptable salt thereof.

Embodiments described herein relate to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the amounts together are effective in treating non-small cell lung cancer.

Additional embodiments described herein relate to a method for treating non-small cell lung cancer comprising administering to a patient in need thereof a synergistic amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, in combination with palbociclib, or a pharmaceutically acceptable salt thereof.

Further embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of non-small cell lung cancer.

Some embodiments described herein relate to a use of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of non-small cell lung cancer.

Additional embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of non-small cell lung cancer, wherein the combination is synergistic.

Some embodiments described herein relate to a use of a synergistic amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of non-small cell lung cancer.

In embodiments of the method or use of the present invention, the non-small cell lung cancer is EGFR-mutant non-small cell lung cancer.

Embodiments described herein relate to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the amounts together are effective in treating EGFR-mutant non-small cell lung cancer.

Additional embodiments described herein relate to a method for treating EGFR-mutant non-small cell lung cancer comprising administering to a patient in need thereof a synergistic amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, in combination with palbociclib, or a pharmaceutically acceptable salt thereof.

Further embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of EGFR-mutant non-small cell lung cancer.

Some embodiments described herein relate to a use of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of EGFR-mutant non-small cell lung cancer.

Additional embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of EGFR-mutant non-small cell lung cancer, wherein the combination is synergistic.

Some embodiments described herein relate to a use of a synergistic amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of EGFR-mutant non-small cell lung cancer.

In embodiments of the method or use of the present invention, the EGFR-mutant non-small cell lung cancer includes a mutation selected from the group consisting of del 19, L858R, del 19/T790M and L858R/T790M.

In embodiments of the method or use of the present invention, the EGFR-mutant non-small cell lung cancer includes a mutation selected from the group consisting of del 19 and L858R.

In embodiments of the method or use of the present invention, the EGFR-mutant non-small cell lung cancer includes a mutation selected from the group consisting of del 19/T790M and L858R/T790M.

In embodiments of the method or use of the present invention, the EGFR-mutant non-small cell lung cancer is advanced EGFR-mutant non-small cell lung cancer.

In embodiments of the method or use of the present invention, the advanced EGFR-mutant non-small cell lung cancer includes a mutation selected from the group consisting of del 19, L858R, del 19/T790M and L858R/T790M.

In embodiments of the method or use of the present invention, the advanced EGFR-mutant non-small cell lung cancer includes a mutation selected from the group consisting of del 19 and L858R.

In embodiments of the method or use of the present invention, the advanced EGFR-mutant non-small cell lung cancer includes a mutation selected from the group consisting of del 19/T790M and L858R/T790M.

Embodiments described herein relate to a method for treating non-small cell lung cancer comprising administering to a patient in need thereof an amount of an EGFR T790M inhibitor and an amount of a CDK inhibitor, wherein the CDK inhibitor is administered according to a non-standard clinical dosing regimen, and further wherein the amounts together are effective in treating non-small cell lung cancer.

Further embodiments described herein relate to a combination of an EGFR T790M inhibitor and a CDK inhibitor, for use in the treatment of non-small cell lung cancer, wherein the CDK inhibitor is administered according to a non-standard clinical dosing regimen.

Additional embodiments described herein relate to a use of an EGFR T790M inhibitor and a CDK inhibitor, in the manufacture of a medicament for the treatment of non-small cell lung cancer, wherein the CDK inhibitor is administered according to a non-standard clinical dosing regimen.

In embodiments of the method or use of the present invention, the non-standard clinical dosing regimen is a non-standard clinical dose.

In embodiments of the method or use of the present invention, the non-standard clinical dose is a low-dose amount of the CDK inhibitor.

In embodiments of the method or use of the present invention, the non-standard clinical dosing regimen is a non-standard dosing schedule.

In embodiments of the method or use of the present invention, the non-standard dosing schedule is a continuous dosing schedule of the CDK inhibitor.

In embodiments of the method or use of the present invention, the EGFR T790M inhibitor is irreversible.

In embodiments of the method or use of the present invention, the EGFR T790M inhibitor is selected from the group consisting of osimertinib, olmutinib, naquotinib, nazartinib, rociletinib, WZ4002 and TAS-2913, or a pharmaceutically acceptable salt thereof.

In embodiments of the method or use of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof.

In embodiments of the method or use of the present invention, the CDK inhibitor is a CDK 4/6 inhibitor.

In embodiments of the method or use of the present invention, the CDK 4/6 inhibitor is selected from the group consisting of abemaciclib, ribociclib and palbociclib, or a pharmaceutically acceptable salt thereof.

In embodiments of the method or use of the present invention, the CDK 4/6 inhibitor is palbociclib, or a pharmaceutically acceptable salt thereof.

In embodiments of the method or use of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and the CDK inhibitor is palbociclib, or a pharmaceutically acceptable salt thereof.

Embodiments described herein relate to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein palbociclib, or a pharmaceutically acceptable salt thereof, is administered according to a non-standard clinical dosing regimen, and further wherein the amounts together are effective in treating non-small cell lung cancer.

Further embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of non-small cell lung cancer, wherein palbociclib, or a pharmaceutically acceptable salt thereof, is administered according to a non-standard clinical dosing regimen.

Additional embodiments described herein relate to a use of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of non-small cell lung cancer, wherein palbociclib, or a pharmaceutically acceptable salt thereof, is administered according to a non-standard clinical dosing regimen.

In embodiments of the method or use of the present invention, the non-standard clinical dosing regimen is a non-standard clinical dose.

In embodiments of the method or use of the present invention, the non-standard clinical dose is a low-dose amount of palbociclib, or a pharmaceutically acceptable salt thereof.

In embodiments of the method or use of the present invention, the low-dose amount of palbociclib, or a pharmaceutically acceptable salt thereof, is about 50 mg, about 75 mg or about 100 mg once daily.

In embodiments of the method or use of the present invention, the low-dose amount of palbociclib, or a pharmaceutically acceptable salt thereof, is about 75 mg once daily.

In embodiments of the method or use of the present invention, the low-dose amount of palbociclib, or a pharmaceutically acceptable salt thereof, is about 100 mg once daily.

In embodiments of the method or use of the present invention, the non-standard clinical dosing regimen is a non-standard dosing schedule.

In embodiments of the method or use of the present invention, the non-standard dosing schedule is a continuous dosing schedule of palbociclib, or a pharmaceutically acceptable salt thereof.

In embodiments of the method or use of the present invention, the continuous dosing schedule of palbociclib, or a pharmaceutically acceptable salt thereof, is a complete cycle of 21 days.

In embodiments of the method or use of the present invention, the continuous dosing schedule of palbociclib, or a pharmaceutically acceptable salt thereof, is a complete cycle of 28 days.

In embodiments of the method or use of the present invention, the non-standard dosing schedule comprises administering palbociclib, or a pharmaceutically acceptable salt thereof, once daily for 14 consecutive days followed by 7 days off treatment.

In embodiments of the method or use of the present invention, the non-standard clinical dosing regimen comprises administering about 75 mg of palbociclib, or a pharmaceutically acceptable salt thereof, once daily for 14 consecutive days followed by 7 days off treatment.

In embodiments of the method or use of the present invention, the non-small cell lung cancer is EGFR-mutant non-small cell lung cancer.

Embodiments described herein relate to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein palbociclib, or a pharmaceutically acceptable salt thereof, is administered according to a non-standard clinical dosing regimen, and further wherein the amounts together are effective in treating EGFR-mutant non-small cell lung cancer.

Further embodiments described herein relate to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of EGFR-mutant non-small cell lung cancer, wherein palbociclib, or a pharmaceutically acceptable salt thereof, is administered according to a non-standard clinical dosing regimen.

Additional embodiments described herein relate to a use of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, in the manufacture of a medicament for the treatment of EGFR-mutant non-small cell lung cancer, wherein palbociclib, or a pharmaceutically acceptable salt thereof, is administered according to a non-standard clinical dosing regimen.

In embodiments of the method or use of the present invention, the EGFR-mutant non-small cell lung cancer includes a mutation selected from the group consisting of del 19, L858R, del 19/T790M and L858R/T790M.

In embodiments of the method or use of the present invention, the EGFR-mutant non-small cell lung cancer includes a mutation selected from the group consisting of del 19 and L858R.

In embodiments of the method or use of the present invention, the EGFR-mutant non-small cell lung cancer includes a mutation selected from the group consisting of del 19/T790M and L858R/T790M.

In embodiments of the method or use of the present invention, the EGFR-mutant non-small cell lung cancer is advanced EGFR-mutant non-small cell lung cancer.

In embodiments of the method or use of the present invention, the advanced EGFR-mutant non-small cell lung cancer includes a mutation selected from the group consisting of del 19, L858R, del 19/T790M and L858R/T790M.

In embodiments of the method or use of the present invention, the advanced EGFR-mutant non-small cell lung cancer includes a mutation selected from the group consisting of del 19 and L858R.

In embodiments of the method or use of the present invention, the advanced EGFR-mutant non-small cell lung cancer includes a mutation selected from the group consisting of del 19/T790M and L858R/T790M.

Embodiments described herein relate to a synergistic combination of

(a) an EGFR T790M inhibitor; and

(b) a CDK inhibitor.

Further embodiments described herein relate to a synergistic combination of

(a) an EGFR T790M inhibitor; and

(b) a CDK inhibitor,

wherein component (a) and component (b) are synergistic.

Additional embodiments, relate to a pharmaceutical composition of an EGFR T790M inhibitor and a pharmaceutical composition of a CDK inhibitor for use in the treatment of non-small cell lung cancer.

In embodiments of combination of the present invention, the EGFR T790M inhibitor is irreversible.

In embodiments of combination of the present invention, the EGFR T790M inhibitor is selected from the group consisting of osimertinib, olmutinib, naquotinib, nazartinib, rociletinib, WZ4002 and TAS-2913, or a pharmaceutically acceptable salt thereof.

In embodiments of combination of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof.

In embodiments of combination of the present invention, the CDK inhibitor is a CDK 4/6 inhibitor.

In embodiments of combination of the present invention, the CDK 4/6 inhibitor is selected from the group consisting of abemaciclib, ribociclib and palbociclib, or a pharmaceutically acceptable salt thereof.

In embodiments of combination of the present invention, the CDK 4/6 inhibitor is palbociclib, or a pharmaceutically acceptable salt thereof.

In embodiments of combination of the present invention, the EGFR T790M inhibitor is N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and the CDK inhibitor is palbociclib, or a pharmaceutically acceptable salt thereof.

### BRIEF DESCRIPTION OF THE DRAWINGS

FIG. 1 shows the duration of response (“DOR”) in the H1975 cell line for PF-06747775 (“PF7775”) in combination with palbociclib (“Palbo”).

FIG. 2 shows the results of the xenograft model with H1975 tumor bearing nude/nude female mice, which were randomized, daily and orally dosed with vehicle, PF-06747775, palbociclib, or the combination of PF-06747775 and palbociclib. FIG. 2 graphs the tumor volumes, which were measured 2 to 3 times per week and graphed with mean and standard error of the mean.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “an” excipient includes one or more excipients.

As used herein, the term “about” when used to modify a numerically defined parameter (e.g., the dose of an EGFR T790M inhibitor or a CDK inhibitor) means that the parameter may vary by as much as 10% below or above the stated numerical value for that parameter. For example, a dose of about 5 mg may vary between 4.5 mg and 5.5 mg.

As used herein, terms, including, but not limited to, “agent”, “component”, “composition, “compound”, “substance”, “targeted agent”, “targeted therapeutic agent”, and “therapeutic agent” may be used interchangeably to refer to the compounds of the present invention, specifically an EGFR T790M inhibitor and a CDK inhibitor.

The following abbreviations may be used herein: DMSO (dimethylsulphoxide); FBS (fetal bovine serum); RPMI (Roswell Park Memorial Institute); mpk (mg/kg or mg drug per kg body weight of animal); and w/w (weight per weight).

The members of the human epidermal growth factor receptor/epidermal growth factor receptor (HER/EGFR) family of receptors include EGFR/HER-1, HER2/neu/erbB-2, HER3/erbB-3 and HER4/erbB-4.

EGFR inhibitors effectively inhibit two frequent and mutually exclusive primary activating mutations, L858R and del 19, of EGFR. These common EGFR activating mutations, L858R and del 19, are also referred to as single-mutants or single-mutant forms. Examples of EGFR inhibitors include gefitinib, erlotinib, icotinib, vandetanib, lapatinib, neratinib, afatinib, pelitinib, dacomitinib and canertinib. Monoclonal antibody inhibitors of EGFR, such as cetuximab and panitumumab, are also EGFR inhibitors, as defined in the present invention.

Inhibitors of EGFR may be reversible or irreversible inhibitors. Reversible inhibitors of the tyrosine kinase domain of the EFGR molecule attach to and periodically detach from the receptor. Gefitinib, erlotinib, icotinib, vandetanib and lapatinib are examples of reversible EGFR inhibitors. Irreversible inhibitors of the tyrosine kinase domain of the EFGR molecule bind to EGFR irreversibly. Neratinib, afatinib, pelitinib, dacomitinib and canertinib are examples of irreversible EGFR inhibitors.

EGFR inhibitors are inhibitors of at least one member of the HER family. Gefitinib, erlotinib, icotinib and vandetanib are selective EGFR/HER-1 tyrosine kinase inhibitors (TKI). Cetuximab and panitumumab are monoclonal antibodies specific to EGFR/HER-1.

A pan-HER inhibitor is an agent that block multiple members of the HER family. Lapatinib, neratinib, afatinib, pelitinib, dacomitinib and canertinib are examples of pan-HER inhibitors. Lapatinib, neratinib, afatinib and pelitinib inhibit the EGFR and HER2 members of the HER family. Dacomitinib and canertinib inhibit the EGFR, HER2, and HER4 members of the HER family.

EGFR T790M inhibitors effectively inhibit the common activating mutations (L858R and del 19) and the gatekeeper mutation (T790M). For purposes of the present invention, the term “L858R/T790M” means L858R and T790M and the term “del 19/T790M” means del 19 and T790M. L858R/T790M and del 19/T790M are referred to as double-mutants, double-mutant variants or double-mutant forms of EGFR.

Inhibitors of EGFR T790M may be reversible or irreversible inhibitors. Brigatinib, PKC412 and Go6976 are non-limiting examples of reversible EGFR T790M inhibitors. PF-06747775, osimertinib, olmutinib, naquotinib, nazartinib, rociletinib, WZ4002 and TAS-2913 are non-limiting examples irreversible EGFR T790M inhibitors.

In an embodiment, an EGFR T790M inhibitor of the present invention includes PF-06747775 (also referred to herein as “PF-7775”, “PF7775”, and “7775”). PF-06747775 is a potent and irreversible inhibitor against the EGFR double-mutants (L858R/T790M and del 19/T790M) and single-mutants (L858R and del 19) and a weak inhibitor of wild-type EGFR.

Cyclin-dependent kinases (CDKs) and related serine/threonine kinases are important cellular enzymes that perform essential functions in regulating cell division and proliferation. CDK inhibitors include Pan-CDK inhibitors that target a broad spectrum of CDKs or selective CDK inhibitors that target specific CDK(s). CDK inhibitors may have activity against targets in addition to CDKs, such as Aurora A, Aurora B, Chk1, Chk2, ERK1, ERK2, GST-ERK1, GSK-3α, GSK-3β, PDGFR, TrkA and VEGFR. CDK inhibitors include, but are not limited to, abemaciclib, alvocidib, dinaciclib, palbociclib, ribociclib, roscovitine, AT7519, AZD5438, BMS-265246, BMS-387032, BS-181, JNJ-7706621, K03861, MK-8776, P276-00, PHA-793887, R547, RO-3306 and SU 9516. Examples of Pan-CDK inhibitors include, but are not limited to, alvocidib, dinaciclib, roscovitine, AT7519, AZD5438, BMS-387032, P276-00, PHA-793887, R547 and SU 9516. A non-limiting example of a CDK1 inhibitor is RO-3306. Examples of CDK2 inhibitors include, but are not limited to, K03861 and MK-8776. Examples of CDK1/2 inhibitors include, but are not limited to, BMS-265246 and JNJ-7706621. Examples of CDK4/6 inhibitors include, but are not limited to, abemaciclib, ribociclib and palbociclib. A non-limiting example of a CDK7 inhibitor is BS-181.

In an embodiment, CDK4/6 inhibitors of the present invention include palbociclib. Unless otherwise indicated herein, palbociclib (also referred to herein as “palbo” or “Palbo”) to 6-acetyl-8-cyclopentyl-5-methyl-2-(5-piperazin-1-yl-pyridin-2-ylamino)-8H-pyrido[2,3-d]pyrimidin-7-one, or a pharmaceutically acceptable salt thereof.

Some embodiments relate to the pharmaceutically acceptable salts of the compounds described herein. Pharmaceutically acceptable salts of the compounds described herein include the acid addition and base addition salts thereof.

Some embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts.

The compounds described herein that are basic in nature are capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

The term “solvate” is used herein to describe a molecular complex comprising a compound described herein and one or more pharmaceutically acceptable solvent molecules, for example, water and ethanol.

The compounds described herein may also exist in unsolvated and solvated forms. Accordingly, some embodiments relate to the hydrates and solvates of the compounds described herein.

Compounds described herein containing one or more asymmetric carbon atoms can exist as two or more stereoisomers. Where a compound described herein contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds described herein containing, for example, an imino, keto, or oxime group, or so-called valence tautomerism in compounds which contain an aromatic moiety. A single compound may exhibit more than one type of isomerism.

The compounds of the embodiments described herein include all stereoisomers (e.g., cis and trans isomers) and all optical isomers of compounds described herein (e.g., R and S enantiomers), as well as racemic, diastereomeric and other mixtures of such isomers. While all stereoisomers are encompassed within the scope of our claims, one skilled in the art will recognize that particular stereoisomers may be preferred.

In some embodiments, the compounds described herein can exist in several tautomeric forms, including the enol and imine form, and the keto and enamine form and geometric isomers and mixtures thereof. All such tautomeric forms are included within the scope of the present embodiments. Tautomers exist as mixtures of a tautomeric set in solution. In solid form, usually one tautomer predominates. Even though one tautomer may be described, the present embodiments include all tautomers of the present compounds.

Included within the scope of the present embodiments are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

The present embodiments also include atropisomers of the compounds described herein. Atropisomers refer to compounds that can be separated into rotationally restricted isomers.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where a compound described herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

A “patient” to be treated according to this invention includes any warm-blooded animal, such as, but not limited to human, monkey or other lower-order primate, horse, dog, rabbit, guinea pig, or mouse. For example, the patient is human. Those skilled in the medical art are readily able to identify individual patients who are afflicted with non-small cell lung cancer and who are in need of treatment.

“EGFR-mutant” or “EGFRm”, as used herein, as it relates to non-small cell lung cancer, includes single mutations, single mutations that confer sensitivity or resistance to EGFR TKIs, and double mutations that arise de novo or arise in response to TKI therapy (resistance mutations). EGFR-mutants include, but are not limited to, inframe deletions, insertions and point mutations within exons 18 to 21 of the kinase domain of EGFR, as well as exon 18-25 kinase domain duplications (“KDD”) and rearrangements. EGFR mutations include, but are not limited to, del 19, L858R, exon 18 insertions, exon 19 insertions, E709X, G719X, A763_Y764insFQEA, S768I, L861Q, exon 20 insertions, T790M, C797X, EGFR-KDD, EGFR-RAS51 and EGFR-PURB. In an embodiment, EGFR-mutant NSCLC includes single activating mutations del 19 and L858R, and the secondary resistance mutation T790M. In an embodiment, EGFR-mutant NSCLC includes the single-mutants, L858R and del 19. In an embodiment, EGFR-mutant NSCLC includes the single-mutant, L858R. In an embodiment, EGFR-mutant NSCLC includes the single-mutant, del 19. In an embodiment, EGFR-mutant NSCLC includes the EGFR double-mutants, del 19/T790M and L858R/T790M. In an embodiment, EGFR-mutant NSCLC includes the EGFR double-mutant, del 19/T790M. In an embodiment, EGFR-mutant NSCLC includes the EGFR double-mutant, L858R/T790M.

The term “advanced”, as used herein, as it relates to non-small cell lung cancer, includes locally advanced (non-metastatic) disease and metastic disease. Locally advanced NSCLC, which may or may not be be treated with curative intent, and metastatic disease, which cannot be treated with curative intent are included within the scope of “advanced non-small cell lung cancer, as used in the present invention. Those skilled in the art will be able to recognize and diagnose advanced non-small cell lung cancer in a patient.

“Duration of Response” for purposes of the present invention means the time from documentation of tumor model growth inhibition due to drug treatment to the time of acquisition of a restored growth rate similar to pretreatment growth rate.

The term “additive” is used to mean that the result of the combination of two compounds, components or targeted agents is no greater that the sum of each compound, component or targeted agent individually. The term “additive” means that there is no improvement in the disease condition or disorder being treated over the use of each compound, component or targeted agent individually.

The terms “synergy” or “synergistic” are used to mean that the result of the combination of two compounds, components or targeted agents is greater than the sum of each agent together. The terms “synergy” or “synergistic” means that there is an improvement in the disease condition or disorder being treated, over the use of each compound, component or targeted agent individually. This improvement in the disease condition or disorder being treated is a “synergistic effect”. A “synergistic amount” is an amount of the combination of the two compounds, components or targeted agents that results in a synergistic effect, as “synergistic” is defined herein.

Determining a synergistic interaction between one or two components, the optimum range for the effect and absolute dose ranges of each component for the effect may be definitively measured by administration of the components over different w/w ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concertrations required in humans and other species by the application of pharmacokinetic/pharmacodynamic methods.

In accordance with the present invention, an amount of a first compound or component is combined with an amount of a second compound or component, and the amounts together are effective in the treatment of non-small cell lung cancer. The amounts, which together are effective, will relieve to some extent one or more of the symptoms of the disorder being treated. In reference to the treatment of cancer, a effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression”.

In an embodiment, the invention is related to a method for treating non-small cell lung cancer comprising administering to a patient in need thereof an amount of an EGFR T790M inhibitor in combination with an amount of a CDK inhibitor, that is effective in treating non-small cell lung cancer. In a further embodiment, the invention is related to a method for treating non-small cell lung cancer comprising administering to a patient in need thereof an amount of an EGFR T790M inhibitor and an amount of a CDK inhibitor, wherein the amounts together are effective in treating non-small cell lung cancer. In another embodiment, the invention is related to combination of an EGFR T790M inhibitor and a CDK inhibitor, for use in the treatment of non-small cell lung cancer. In another embodiment, the invention is related to a method for treating non-small cell lung cancer comprising administering to a patient in need thereof an amount of an EGFR T790M inhibitor and an amount of a CDK inhibitor, wherein the amounts together achieve synergistic effects in the treatment of non-small cell lung cancer. In another embodiment, the invention is related to a combination of an EGFR T790M inhibitor and a CDK inhibitor for the treatment of non-small cell lung cancer, wherein the combination is synergistic. In an embodiment, the method or use of the invention is related to a synergistic combination of targeted therapeutic agents, specifically an EGFR T790M inhibitor and a CDK inhibitor.

In an embodiment, the invention is related to a method for treating non-small cell lung cancer comprising administering to a patient in need thereof an amount of an irreversible EGFR T790M inhibitor in combination with an amount of a CDK inhibitor, that is effective in treating non-small cell lung cancer. In a further embodiment, the invention is related to a method for treating non-small cell lung cancer comprising administering to a patient in need thereof an amount of an irreversible EGFR T790M inhibitor and an amount of a CDK inhibitor, wherein the amounts together are effective in treating non-small cell lung cancer. In another embodiment, the invention is related to a combination of an irreversible EGFR T790M inhibitor and a CDK inhibitor for use in the treatment of non-small cell lung cancer. In another embodiment, the invention is related to a method for treating non-small cell lung cancer comprising administering to a patient in need thereof an amount of an irreversible EGFR T790M inhibitor and an amount of a CDK inhibitor, wherein the amounts together achieve synergistic effects in the treatment of non-small cell lung cancer. In another embodiment, the invention is related to a combination of an irreversible EGFR T790M inhibitor and a CDK inhibitor for the treatment of non-small cell lung cancer, wherein the combination is synergistic. In an embodiment, the method or use of the invention is related to a synergistic combination of targeted therapeutic agents, specifically an irreversible EGFR T790M inhibitor and a CDK inhibitor.

In an embodiment, the invention is related to a method for treating non-small cell lung cancer comprising administering to a patient in need thereof an amount of an EGFR T790M inhibitor in combination with an amount of a CDK 4/6 inhibitor, that is effective in treating non-small cell lung cancer. In a further embodiment, the invention is related to a method for treating non-small cell lung cancer comprising administering to a patient in need thereof an amount of an EGFR T790M inhibitor and an amount of a CDK 4/6 inhibitor, wherein the amounts together are effective in treating non-small cell lung cancer. In another embodiment, the invention is related to a combination of an EGFR T790M inhibitor and a CDK 4/6 inhibitor in the treatment of non-small cell lung cancer. In another embodiment, the invention is related to a method for treating non-small cell lung cancer comprising administering to a patient in need thereof an amount of an EGFR T790M inhibitor and an amount of a CDK 4/6 inhibitor, wherein the amounts together achieve synergistic effects in the treatment of non-small cell lung cancer. In another embodiment, the invention is related to a combination of an EGFR T790M inhibitor and a CDK 4/6 inhibitor for the treatment of non-small cell lung cancer, wherein the combination is synergistic. In an embodiment, the method or use of the invention is related to a synergistic combination of targeted therapeutic agents, specifically an EGFR T790M inhibitor and a CDK 4/6 inhibitor.

In an embodiment, the invention is related to a method for treating non-small cell lung cancer comprising administering to a patient in need thereof an amount of an irreversible EGFR T790M inhibitor in combination with an amount of a CDK 4/6 inhibitor, that is effective in treating non-small cell lung cancer. In a further embodiment, the invention is related to a method for treating non-small cell lung cancer comprising administering to a patient in need thereof an amount of an irreversible EGFR T790M inhibitor and an amount of a CDK 4/6 inhibitor, wherein the amounts together are effective in treating non-small cell lung cancer. In another embodiment, the invention is related to a combination of an irreversible EGFR T790M inhibitor and a CDK 4/6 inhibitor in the treatment of non-small cell lung cancer. In another embodiment, the invention is related to a method for treating non-small cell lung cancer comprising administering to a patient in need thereof an amount of an irreversible EGFR T790M inhibitor and an amount of a CDK 4/6 inhibitor, wherein the amounts together achieve synergistic effects in the treatment of non-small cell lung cancer. In another embodiment, the invention is related to a combination of an irreversible EGFR T790M inhibitor and a CDK 4/6 inhibitor for the treatment of non-small cell lung cancer, wherein the combination is synergistic. In an embodiment, the method or use of the invention is related to a synergistic combination of targeted therapeutic agents, specifically an irreversible EGFR T790M inhibitor and a CDK 4/6 inhibitor.

In an embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, in combination with an amount of palbociclib, or a pharmaceutically acceptable salt thereof, that is effective in treating non-small cell lung cancer. In a further embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the amounts together are effective in treating non-small cell lung cancer. In another embodiment, the invention is related to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, wherein the amounts together are effective in treating non-small cell lung cancer. In another embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the amounts together achieve synergistic effects in the treatment of non-small cell lung cancer. In another embodiment, the invention is related to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof for the treatment of non-small cell lung cancer, wherein the combination is synergistic. In an embodiment, the method or use of the invention is related to a synergistic combination of targeted therapeutic agents, specifically N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof.

A “standard clinical dosing regimen,” as used herein, refers to a regimen for administering a substance, agent, compound, or composition, which is typically used in a clinical setting. A “standard clinical dosing regimen,” includes a “standard clinical dose” or a “standard dosing schedule”.

A “non-standard clinical dosing regimen,” as used herein, refers to a regimen for administering a substance, agent, compound, or composition, which is different than the amount, dose or schedule typically used in a clinical setting. A “non-standard clinical dosing regimen,” includes a “non-standard clinical dose” or a “non-standard dosing schedule”.

In an embodiment, the invention is related to a method for treating non-small cell lung cancer comprising administering to a patient in need thereof an amount of an EGFR T790M inhibitor in combination with an amount of a CDK inhibitor, that is effective in treating non-small cell lung cancer, wherein the CDK inhibitor is administered according to a non-standard clinical dosing regimen. In a further embodiment, the invention is related to a method for treating non-small cell lung cancer comprising administering to a patient in need thereof an amount of an EGFR T790M inhibitor and an amount of a CDK inhibitor, wherein the CDK inhibitor is administered according to a non-standard clinical dosing regimen, and further wherein the amounts together are effective in treating non-small cell lung cancer. In another embodiment, the invention is related to a combination of an EGFR T790M inhibitor and an amount of a CDK inhibitor for use in the treatment of non-small cell lung cancer, wherein the CDK inhibitor is administered according to a non-standard clinical dosing regimen. In another embodiment, the invention is related to a method for treating non-small cell lung cancer comprising administering to a patient in need thereof an amount of an EGFR T790M inhibitor and an amount of a CDK inhibitor, wherein the CDK inhibitor is administered according to a non-standard clinical dosing regimen, and further wherein the amounts together achieve synergistic effects in treating non-small cell lung cancer. In another embodiment, the invention is related to a combination of an EGFR T790M inhibitor and a CDK inhibitor for the treatment of non-small cell lung cancer, wherein the CDK inhibitor is administered according to a non-standard clinical dosing regimen, and further wherein the combination is synergistic.

In an embodiment, the invention is related to a method for treating non-small cell lung cancer comprising administering to a patient in need thereof an amount of an irreversible EGFR T790M inhibitor in combination with an amount of a CDK inhibitor, that is effective in treating non-small cell lung cancer, wherein the CDK inhibitor is administered according to a non-standard clinical dosing regimen. In a further embodiment, the invention is related to a method for treating non-small cell lung cancer comprising administering to a patient in need thereof an amount of an irreversible EGFR T790M inhibitor and an amount of a CDK inhibitor, wherein the CDK inhibitor is administered according to a non-standard clinical dosing regimen, and further wherein the amounts together are effective in treating non-small cell lung cancer. In another embodiment, the invention is related to the use of a combination of an irreversible EGFR T790M inhibitor and a CDK inhibitor in the treatment of non-small cell lung cancer, wherein the CDK inhibitor is administered according to a non-standard clinical dosing regimen. In another embodiment, the invention is related to a method for treating non-small cell lung cancer comprising administering to a patient in need thereof an amount of an irreversible EGFR T790M inhibitor and an amount of a CDK inhibitor, wherein the CDK inhibitor is administered according to a non-standard clinical dosing regimen, and further wherein the amounts together achieve synergistic effects in treating non-small cell lung cancer. In another embodiment, the invention is related to the use of an amount a combination of an irreversible EGFR T790M inhibitor and a CDK inhibitor for the treatment of non-small cell lung cancer, wherein the CDK inhibitor is administered according to a non-standard clinical dosing regimen.

In an embodiment, the invention is related to a method for treating non-small cell lung cancer comprising administering to a patient in need thereof an amount of an EGFR T790M inhibitor in combination with an amount of a CDK 4/6 inhibitor, that is effective in treating non-small cell lung cancer, wherein the CDK 4/6 inhibitor is administered according to a non-standard clinical dosing regimen. In a further embodiment, the invention is related to a method for treating non-small cell lung cancer comprising administering to a patient in need thereof an amount of an EGFR T790M inhibitor and an amount of a CDK 4/6 inhibitor, wherein the CDK 4/6 inhibitor is administered according to a non-standard clinical dosing regimen, and further wherein the amounts together are effective in treating non-small cell lung cancer. In another embodiment, the invention is related to a combintaion of an EGFR T790M inhibitor and a CDK 4/6 inhibitor in the treatment of non-small cell lung cancer, wherein the CDK 4/6 inhibitor is administered according to a non-standard clinical dosing regimen. In another embodiment, the invention is related to a method for treating non-small cell lung cancer comprising administering to a patient in need thereof an amount of an EGFR T790M inhibitor and an amount of a CDK 4/6 inhibitor, wherein the CDK 4/6 inhibitor is administered according to a non-standard clinical dosing regimen, and further wherein the amounts together achieve synergistic effects in treating non-small cell lung cancer. In another embodiment, the invention is related to a combination of an EGFR T790M inhibitor and a CDK 4/6 inhibitor for the treatment of non-small cell lung cancer, wherein the CDK 4/6 inhibitor is administered according to a non-standard clinical dosing regimen, and further wherein the combination is synergistic.

In an embodiment, the invention is related to a method for treating non-small cell lung cancer comprising administering to a patient in need thereof an amount of an irreversible EGFR T790M inhibitor in combination with an amount of a CDK 4/6 inhibitor, that is effective in treating non-small cell lung cancer, wherein the CDK 4/6 inhibitor is administered according to a non-standard clinical dosing regimen. In a further embodiment, the invention is related to a method for treating non-small cell lung cancer comprising administering to a patient in need thereof an amount of an irreversible EGFR T790M inhibitor and an amount of a CDK 4/6 inhibitor, wherein the CDK 4/6 inhibitor is administered according to a non-standard clinical dosing regimen, and further wherein the amounts together are effective in treating non-small cell lung cancer. In another embodiment, the invention is related to a combination of an irreversible EGFR T790M inhibitor and an amount of a CDK 4/6 inhibitor for use in the treatment of non-small cell lung cancer, wherein the CDK 4/6 inhibitor is administered according to a non-standard clinical dosing regimen. In another embodiment, the invention is related to a method for treating non-small cell lung cancer comprising administering to a patient in need thereof an amount of an irreversible EGFR T790M inhibitor and an amount of a CDK 4/6 inhibitor, wherein the CDK 4/6 inhibitor is administered according to a non-standard clinical dosing regimen, and further wherein the amounts together achieve synergistic effects in treating non-small cell lung cancer. In another embodiment, the invention is related to a combination of an irreversible EGFR T790M inhibitor and a CDK 4/6 inhibitor for the treatment of non-small cell lung cancer, wherein the CDK 4/6 inhibitor is administered according to a non-standard clinical dosing regimen, and further wherein the combination is synergistic.

In an embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, in combination with an amount of palbociclib, or a pharmaceutically acceptable salt thereof, that is effective in treating non-small cell lung cancer, wherein the palbociclib, or a pharmaceutically acceptable salt thereof is administered according to a non-standard clinical dosing regimen. In a further embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of a palbociclib, or a pharmaceutically acceptable salt thereof, wherein the palbociclib, or a pharmaceutically acceptable salt thereof is administered according to a non-standard clinical dosing regimen, and further wherein the amounts together are effective in treating non-small cell lung cancer. In another embodiment, the invention is related to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of non-small cell lung cancer, wherein the palbociclib, or a pharmaceutically acceptable salt thereof is administered according to a non-standard clinical dosing regimen. In another embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the palbociclib, or a pharmaceutically acceptable salt thereof is administered according to a non-standard clinical dosing regimen, and further wherein the amounts together achieve synergistic effects in treating non-small cell lung cancer. In another embodiment, the invention is related to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for the treatment of non-small cell lung cancer, wherein the palbociclib, or a pharmaceutically acceptable salt thereof is administered according to a non-standard clinical dosing regimen, and further wherein the combination is synergistic.

A “low-dose amount”, as used herein, refers to an amount or dose of a substance, agent, compound, or composition, that is lower than the amount or dose typically used in a clinical setting.

In an embodiment, the invention is related to a method for treating non-small cell lung cancer comprising administering to a patient in need thereof an amount of an EGFR T790M inhibitor in combination with a low-dose amount of a CDK inhibitor, that is effective in treating non-small cell lung cancer. In a further embodiment, the invention is related to a method for treating non-small cell lung cancer comprising administering to a patient in need thereof an amount of an EGFR T790M inhibitor and a low-dose amount of a CDK inhibitor, wherein the amounts together are effective in treating non-small cell lung cancer. In another embodiment, the invention is related to a combination of an EGFR T790M inhibitor and a low-dose amount of a CDK inhibitor for use in the treatment of non-small cell lung cancer. In another embodiment, the invention is related to a method for treating non-small cell lung cancer comprising administering to a patient in need thereof an amount of an EGFR T790M inhibitor and a low-dose amount of a CDK inhibitor, wherein the amounts together achieve synergistic effects in the treatment of non-small cell lung cancer. In another embodiment, the invention is related to a combination of an EGFR T790M inhibitor and a low-dose amount of a CDK inhibitor for the treatment of non-small cell lung cancer, wherein the combination is synergistic.

In an embodiment, the invention is related to a method for treating non-small cell lung cancer comprising administering to a patient in need thereof an amount of an irreversible EGFR T790M inhibitor in combination with a low-dose amount of a CDK inhibitor, that is effective in treating non-small cell lung cancer. In a further embodiment, the invention is related to a method for treating non-small cell lung cancer comprising administering to a patient in need thereof an amount of an irreversible EGFR T790M inhibitor and a low-dose amount of a CDK inhibitor, wherein the amounts together are effective in treating non-small cell lung cancer. In another embodiment, the invention is related to a combination of an irreversible EGFR T790M inhibitor and a low-dose amount of a CDK inhibitor for use in the treatment of non-small cell lung cancer. In another embodiment, the invention is related to a method for treating non-small cell lung cancer comprising administering to a patient in need thereof an amount of an irreversible EGFR T790M inhibitor and a low-dose amount of a CDK inhibitor, wherein the amounts together achieve synergistic effects in the treatment of non-small cell lung cancer. In another embodiment, the invention is related to a combination of of an irreversible EGFR T790M inhibitor and a low-dose amount of a CDK inhibitor for the treatment of non-small cell lung cancer, wherein the combination is synergistic.

In an embodiment, the invention is related to a method for treating non-small cell lung cancer comprising administering to a patient in need thereof an amount of an EGFR T790M inhibitor in combination with a low-dose amount of a CDK 4/6 inhibitor, that is effective in treating non-small cell lung cancer. In a further embodiment, the invention is related to a method for treating non-small cell lung cancer comprising administering to a patient in need thereof an amount of an EGFR T790M inhibitor and a low-dose amount of a CDK 4/6 inhibitor, wherein the amounts together are effective in treating non-small cell lung cancer. In another embodiment, the invention is related to a combination of an EGFR T790M inhibitor and a low-dose amount of a CDK 4/6 inhibitor for use in the treatment of non-small cell lung cancer. In another embodiment, the invention is related to a method for treating non-small cell lung cancer comprising administering to a patient in need thereof an amount of an EGFR T790M inhibitor and a low-dose amount of a CDK 4/6 inhibitor, wherein the amounts together achieve synergistic effects in the treatment of non-small cell lung cancer. In another embodiment, the invention is related to a combination of an EGFR T790M inhibitor and a low-dose amount of a CDK 4/6 inhibitor for the treatment of non-small cell lung cancer, wherein the combination is synergistic.

In an embodiment, the invention is related to a method for treating non-small cell lung cancer comprising administering to a patient in need thereof an amount of an irreversible EGFR T790M inhibitor in combination with a low-dose amount of a CDK 4/6 inhibitor, that is effective in treating non-small cell lung cancer. In a further embodiment, the invention is related to a method for treating non-small cell lung cancer comprising administering to a patient in need thereof an amount of an irreversible EGFR T790M inhibitor and a low-dose amount of a CDK 4/6 inhibitor, wherein the amounts together are effective in treating non-small cell lung cancer. In another embodiment, the invention is related to a combination of an irreversible EGFR T790M inhibitor and a low-dose amount of a CDK 4/6 inhibitor for use in the treatment of non-small cell lung cancer. In another embodiment, the invention is related to a method for treating non-small cell lung cancer comprising administering to a patient in need thereof an amount of an irreversible EGFR T790M inhibitor and a low-dose amount of a CDK 4/6 inhibitor, wherein the amounts together achieve synergistic effects in the treatment of non-small cell lung cancer. In another embodiment, the invention is related to a combination of an irreversible EGFR T790M inhibitor and a low-dose amount of a CDK 4/6 inhibitor for the treatment of non-small cell lung cancer, wherein the combination is synergistic.

In an embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, in combination with a low-dose amount of palbociclib, or a pharmaceutically acceptable salt thereof, that is effective in treating non-small cell lung cancer. In a further embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and a low-dose amount of palbociclib, or a pharmaceutically acceptable salt thereof, wherein the amounts together are effective in treating non-small cell lung cancer. In another embodiment, the invention is related to a combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and a low-dose amount of a palbociclib, or a pharmaceutically acceptable salt thereof, for use in the treatment of non-small cell lung cancer. In another embodiment, the invention is related to a method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and a low-dose amount of a palbociclib, or a pharmaceutically acceptable salt thereof, wherein the amounts together achieve synergistic effects in the treatment of non-small cell lung cancer. In another embodiment, the invention is related to a combination of an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and a low-dose amount of palbociclib, or a pharmaceutically acceptable salt thereof, for the treatment of non-small cell lung cancer, wherein the combination is synergistic.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the non-small cell lung cancer requiring treatment, and the presence of other medications.

In an embodiment, PF-06747775, or a pharmaceutically acceptable salt thereof, is administered at a daily dosage of from about 5 mg to about 650 mg once a day, preferably from about 25 mg to about 450 mg once a day, and more preferably from about 150 mg to about 350 mg once a day. In an embodiment, PF-06747775 is administered at a daily dosage of about 50 mg, about 100 mg, about 150 mg or about 200 mg once daily. In an embodiment, PF-06747775 is administered at a daily dosage of about 50 mg once daily. In an embodiment, PF-06747775 is administered at a daily dosage of about 100 mg once daily. In an embodiment, PF-06747775 is administered at a daily dosage of about 150 mg once daily. In an embodiment, PF-06747775 is administered at a daily dosage of about 200 mg once daily. Dosage amounts provided herein refer to the dose of the free base form of PF-06747775, or are calculated as the free base equivalent of an administered PF-06747775 salt form. For example, a dosage or amount of PF-06747775, such as 100 mg, 75 mg or 50 mg, refers to the free base equivalent. This dosage regimen may be adjusted to provide the optimal therapeutic response. For example, the dose may be proportionally reduced or increased as indicated by the exigencies of the therapeutic situation.

In an embodiment, palbociclib, or a pharmaceutically acceptable salt thereof, is administered at a daily dosage of about 125 mg once daily, about 100 mg once daily, about 75 mg once daily, or about 50 mg daily. In an embodiment, which is the recommended starting dose or standard clinical dose, palbociclib, or a pharmaceutically acceptable salt thereof, is administered at a daily dosage of about 125 mg once a day. In an embodiment, palbociclib, or a pharmaceutically acceptable salt thereof, is administered at a non-standard clinical dose. In an embodiment, a non-standard clinical dose is a low-dose amount of palbociclib, or a pharmaceutically acceptable salt thereof. For example, palbociclib, or a pharmaceutically acceptable salt thereof, is administered at a dose of about 100 mg once daily, about 75 mg once daily, or about 50 mg once daily. In an embodiment, palbociclib, or a pharmaceutically acceptable salt thereof, is administered at a dose of about 100 mg once daily. In an embodiment, palbociclib, or a pharmaceutically acceptable salt thereof, is administered at a dose of about 75 mg once daily. In an embodiment, palbociclib, or a pharmaceutically acceptable salt thereof, is administered at a dose of about 50 mg once daily. Dosage amounts provided herein refer to the dose of the free base form of palbociclib, or are calculated as the free base equivalent of an administered palbociclib salt form. For example, a dosage or amount of palbociclib, such as 100 mg, 75 mg or 50 mg, refers to the free base equivalent. This dosage regimen may be adjusted to provide the optimal therapeutic response. For example, the dose may be proportionally reduced or increased as indicated by the exigencies of the therapeutic situation.

The practice of the method of this invention may be accomplished through various administration or dosing regimens. The compounds of the combination of the present invention can be administered intermittently, concurrently or sequentially. In an embodiment, the compounds of the combination of the present invention can be administered in a concurrent dosing regimen.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In an embodiment, PF-06747775, or a pharmaceutically acceptable salt thereof, is administered once daily to comprise a complete cycle of 28 days. Repetition of the 28 day cycles is continued during treatment with the combination of the present invention.

In an embodiment, PF-06747775, or a pharmaceutically acceptable salt thereof, is administered once daily to comprise a complete cycle of 21 days. Repetition of the 21 day cycles is continued during treatment with the combination of the present invention.

The standard recommended dosing regimen, which includes the standard dosing schedule, for palbociclib, or a pharmaceutically acceptable salt thereof, is administration once daily for 21 consecutive days followed by 7 days off treatment to comprise a complete cycle of 28 days. Repetition of the 28 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily to comprise a complete cycle of 28 days. Repetition of the 28 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily to comprise a complete cycle of 21 days. Repetition of the 21 day cycles is continued during treatment with the combination of the present invention.

In an embodiment, palbociclib, or a pharmaceutically acceptable salt thereof, is administered under a non-standard dosing schedule. For example, palbociclib, or a pharmaceutically acceptable salt thereof, is administered once daily for 14 consecutive days followed by 7 days off treatment to comprise a complete cycle of 21 days. Repetition of the 21 day cycles is continued during treatment with the combination of the present invention.

The standard clinical dosing regimen, for palbociclib, or a pharmaceutically acceptable salt thereof, is administration of 125 mg once daily for 21 consecutive days followed by 7 days off treatment to comprise a complete cycle of 28 days. Repetition of the 28 day cycles is continued during treatment with the combination of the present invention.

In an embodiment, palbociclib, or a pharmaceutically acceptable salt thereof, is administered under a non-standard clinical dosing regimen. For example, palbociclib, or a pharmaceutically acceptable salt thereof, is administered at about 50 mg, about 75 mg or about 100 mg once daily to comprise a complete cycle of 28 days. Repetition of the 28 day cycles is continued during treatment with the combination of the present invention. In an embodiment, palbociclib, or a pharmaceutically acceptable salt thereof, is administered at about 50 mg. In an embodiment, palbociclib, or a pharmaceutically acceptable salt thereof, is administered at about 75 mg. In an embodiment, palbociclib, or a pharmaceutically acceptable salt thereof, is administered at about 100 mg.

In an embodiment, palbociclib, or a pharmaceutically acceptable salt thereof, is administered under a non-standard clinical dosing regimen. For example, palbociclib, or a pharmaceutically acceptable salt thereof, is administered at about 50 mg, about 75 mg or about 100 mg once daily to comprise a complete cycle of 21 days. Repetition of the 21 day cycles is continued during treatment with the combination of the present invention. In an embodiment, palbociclib, or a pharmaceutically acceptable salt thereof, is administered at about 50 mg. In an embodiment, palbociclib, or a pharmaceutically acceptable salt thereof, is administered at about 75 mg. In an embodiment, palbociclib, or a pharmaceutically acceptable salt thereof, is administered at about 100 mg.

In an embodiment, palbociclib, or a pharmaceutically acceptable salt thereof, is administered under a non-standard clinical dosing regimen. For example, palbociclib, or a pharmaceutically acceptable salt thereof, is administered at about 75 mg once daily for 14 consecutive days followed by 7 days off treatment to comprise a complete cycle of 21 days. Repetition of the 21 day cycles is continued during treatment with the combination of the present invention.

Administration of the compounds of the combination of the present invention can be effected by any method that enables delivery of the compounds to the site of action. These methods include oral routes, intraduodenal routes, parenteral injection (including intravenous, subcutaneous, intramuscular, intravascular or infusion), topical, and rectal administration.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

### EXAMPLES

### Example 1

### Generation of PF-06747775 Resistant Cell Lines and Utilization in Duration of Response Experiments

Six cell lines of EGFR-mutant NSCLC were evaluated. The H1975 cell line harbors the activating mutation L858R and the erlotinib/gefitinib resistant mutation T790M, with both mutations on the same allele, representing the second line T790M patient population that has progressed on an initial EGFR TKI therapy. The other five cell lines represent the first line EGFR-mutant patient population. The H3255 cell line harbors the activating mutation L858R. The HCC4006, HCC827, PC9, and HCC2935 cell lines each harbor the activating mutation designated del 19, which is a short in-frame deletion in exon 19.

H1975, HCC4006, HCC827, and HCC2935 were purchased from ATCC (Manassas, Va., USA) and were cultured according to ATCC recommendations. PC9 cells were purchased from RIKEN Cell Bank (Tsukuba, Ibaraki Prefecture, Japan) and were cultured in Gibco RPMI 1640 (Life Technologies, Carlsbad, Calif., USA) medium with 10% FBS (Sigma, St. Louis, Mo., USA). H3255 cells were from Dr. Bruce E. Johnson at the National Cancer Institute (Bethesda, Md., USA) and were cultured in RPMI 1640, 10% FBS, and ACL-4 supplement (Mediatech Inc, Manassas, Va., USA).

Each of the cell lines growing in culture was treated with 600 nM PF-06747775, a concentration which approximates a clinically acheivable exposure. For each of the cell lines, treatment with PF-06747775 led to pronounced inhibition of cell viability and growth, within a few days. Treatment with 600 nM PF-06747775 was refreshed weekly and was maintained continuously for weeks to months, until actively growing cells re-emerged. This re-emergence of growing cells in the presence of PF-06747775 represented the acquisition of resistance or selection of pre-existing cells resistant to PF-06747775, as has been well-documented in the literature for similar studies with erlotinib, gefitinib, and other EGFR TKIs in these same models (Koizumi F., et al., “Establishment of a human non-small cell lung cancer cell line resistant to gefitinib,” International Journal of Cancer, 2005, 36-44, vol. 116, no. 1; Engelman, J., et al., “Allele dilution obscures detection of a biologically significant resistance mutation in EGFR-amplified lung cancer,” Journal of Clinical Investigation, 2006, 2695-2706, vol. 10; Ercan, D., et al., “Amplification of EGFR T790M causes resistance to an irreversible EGFR inhibitor,” Oncogene, 2010, 2346-2356, vol. 29, no. 16; Chmielecki, J., et al, “Optimization of dosing for EGFR-mutant non-small cell lung cancer with evolutionary cancer modeling,” Science Translational Medicine, 2011, 90ra59, vol. 3, no. 90). PF-06747775-resistant cells emerged for four of the six models (H1975, HCC827, PC9, and HCC4006). While the time to emergence of resistant cell growth varied between the four cell lines, it was reproducible and consistent across independent experiments in each cell line. Over the course of several months of experimentation, the remaining two cell lines (H3255 and HCC2935) did not give rise to PF-06747775 resistant growth.

The four cell lines that reproducibly gave rise to resistance were used in subsequent studies to test whether the combination of palbociclib with PF-06747775 could delay the emergence of resistance. This experimental approach provides a model of DOR and a means for testing whether combination regimens can increase or extend DOR (Tricker E M, Xu C, Uddin S, et al. Combined EGFR/MEK Inhibition Prevents the Emergence of Resistance in EGFR-Mutant Lung Cancer. Cancer Discov 2015; 5(9):960-71). Such DOR or time to resistance studies were conducted using palbociclib at a concentration of 100 nM, an approximation of clinical free drug exposure at the approved dose. PF-06747775 was used at 600 nM, and the two drugs were tested as single agents or in combination. Two different assay formats were used to facilitate quantitative assessment of time to regrowth and to address reproducibility in orthogonal formats.

### Example 2

### Duration of Response Study for PF-06747775 and Palbociclib, Alone and in Combination, in H1975 Cell Line Model (T75 Flask Format)

H1975 cells were seeded (5×10⁵ cells per flask) in T75 flasks (one or two flasks per treatment condition). When actively growing cells reached 50% confluence, treatment was initiated with PF-06747775 at 600 nM and palbociclib at 100 nM, a concentration which approximates a clinically acheivable exposure. PF-06747775 and palbociclib were tested as single agents and in combination. Treatments with PF-06747775 and palbociclib were refreshed weekly. Time was monitored. When growth reached 70-90% confluence, the cells were harvested, counted, and half of the collected cells were used to seed a fresh T75 flask. Once growth reached 70-90% confluence again, this process was repeated, then repeated again and again until a constant growth rate was achieved. Results were plotted as total live cells counted over time, for example, at each harvest, the cell number counted at that harvest was added to previous cell count numbers, then plotted as total live cells at that time point.

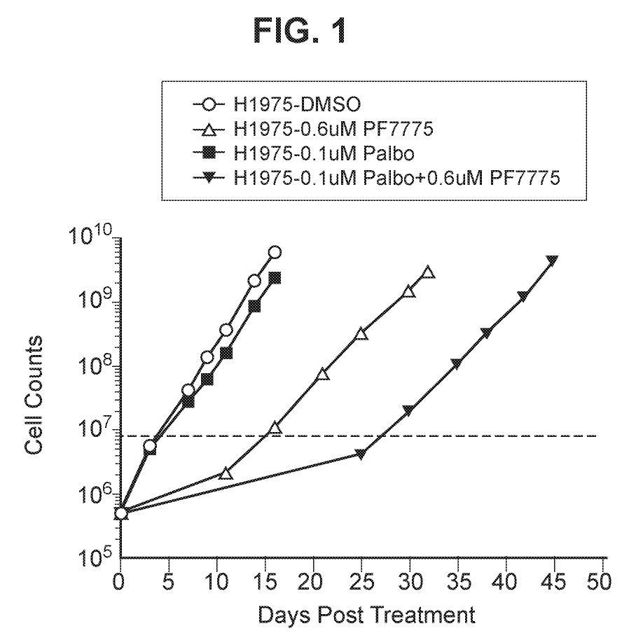

The DOR results for the H1975 cell line using the T75 flask format are shown in FIG. 1. The time required to reach a specific total cell number was least with DMSO treated cells, which served as the control for this study and represented the non-drug-impeded growth rate. Single agent palbociclib treatment showed a slight delay, if any, compared to the control. Single agent PF-06747775 showed a significant delay, requiring more time to reach the same number of cells as the control, representing the time required for resistant growth to emerge. The combination of PF-06747775 and palbociclib showed the greatest delay, representing further impedance of time to resistance with the combination.

By selecting a starting cell count number of 5×10⁵ cells and an ending cell count number of 8×10⁶ cells, Table 1 shows the results of two independent experiments that determined the number of days it took for 5×10⁵ cells to grow to 8×10⁶ cells for each of the different treatment conditions.

TABLE 1
H1975 Duration of Response Study in T75 Flask Format

Independent Experiment

| | Number 1 | Number 2 |
|---|---|---|
| Treatment Condition | (days) | (days) |
| control | 4 | 4 |
| PF-06747775 | 15 | 21 |
| palbociclib | 4 | 5 |
| PF-06747775 and palbociclib | 28 | 29 |

In conclusion, the combination of PF-06747775 and palbociclib increased duration of response as compared to single agent treatment, in the H1975 cell line, which is a NSCLC cell line model representing the second line T790M patient population that has progressed on an initial EGFR TKI therapy.

### Example 3

### Duration of Response Study for PF-06747775 and Palbociclib, Alone and in Combination, in H1975 Cell Line Model (96-Well Plate Format)

A 96-well plate format assay was adapted from a published report (Tricker, E., et al., “Combined EGFR/MEK Inhibition Prevents the Emergence of Resistance in EGFR-Mutant Lung Cancer,” Cancer Discovery, 2015, 960-971, vol. 5, no. 9). Several (two to six) 96-well plates per treatment condition were seeded with 350 H1975 cells per well, then treated with PF-06747775 at 600 nM or palbociclib at 100 nM as single agents or in combination. Time was monitored. Treatments with PF-06747775 and palbociclib were refreshed weekly. Non-terminal reading of live cells was conducted weekly using an IncuCyte instrument (microscopy-based cell counting, Essen Bioscience, Ann Arbor, Mich., USA). At selected time points throughout the treatment duration, a terminal reading of live cells by Cell Titer Glow method (cell ATP-based counting, Promega, Madison, Wis., USA) was used to confirm cell counts over time and at the end of study. Results were plotted as percent of wells reaching a designated confluency (e.g. 50% confluent) at different time points throughout the treatment period.

Table 2 shows the results of two independent experiments that determined the number of days it took for 350 cells per well initially to grow to 50% of the wells reaching 50% confluence, for each of the different treatment conditions.

TABLE 2
H1975 Duration of Response Study in 96-Well Plate Format

Independent Experiment

| | Number 1 | Number 2 |
|---|---|---|
| Treatment Condition | (days) | (days) |
| PF-06747775 | 16 | 19 |
| palbociclib | 7 | 7 |
| PF-06747775 and palbociclib | 40 | 24 |

In conclusion, the combination of PF-06747775 and palbociclib increased duration of response as compared to single agent treatment, in the H1975 cell line, which is a NSCLC cell line model representing the second line T790M patient population that has progressed on an initial EGFR TKI therapy.

### Example 4

### Duration of Response Study for PF-06747775 in Combination with Palbociclib at Lower Concentrations, in H1975 Cell Line Model (T75 Flask Format)

Using the method of Example 2, lower concentrations of palbociclib were tested in combination with PF-06747775 in duration of response experiments in the H1975 cell line (Table 3).

TABLE 3
H1975 Duration of Response Study of Lower Palbociclib
Concentrations in T75 Flask Format

| Treatment Condition | Time Elapsed (days) |
|---|---|
| DMSO | 4 |
| 100 nM palbociclib | 5 |
| 600 nM PF-06747775 | 21 |
| 600 nM PF-7775 + 100 nM palbo | 29 |
| 600 nM PF-7775 + 75 nM palbo | 29 |
| 600 nM PF-7775 + 50 nM palbo | 29 |
| 600 nM PF-7775 + 25 nM palbo | 27 |

The increased duration of response observed from the combination of PF-06747775 and palbociclib was the same using a fixed concentration of PF-06747775 at 600 nM plus palbociclib at 100 nM, 75 nM, or 50 nM. These results indicate that combination efficacy is maintained at lower concentrations of palbociclib.

### Example 5

### Duration of Response Study for PF-06747775 and Palbociclib, Alone and in Combination, in HCC4006, HCC827 and PC9 Cell Line Models (T75 Flask Format)

Using the method of Example 2, PF-06747775 was tested alone and in combination with palbocicib in duration of response experiments in the cell lines that became resistant to PF-06747775 in Example 1.

Table 4 shows the results of two independent experiments that determined the number of days it took for 5×10⁵ cells to grow to 8×10⁶ cells for each of the different treatment conditions.

TABLE 4
HCC4006, HCC827, and PC9 Duration of
Response Study (T75 Flask Format)

| | | HCC827 (independent | | |
|---|---|---|---|---|
| | PC9 | experiment) | | HCC4006 |
| | Time elapsed | Number 1 | Number 2 | Time elapsed |
| Treatment | (days) | (days) | (days) | (days) |
| DMSO | NA | 8 | NA | 16 |
| Palbo | 5 | 11 | 11 | 18 |
| PF-7775 | 73 | 50 | 20 | 51 |
| PF-7775 + palbo | 121 | 64 | 46 | 78 |

In conclusion, the combination of PF-06747775 and palbociclib increased duration of response as compared to single agent treatment, in the HCC4006, HCC827, PC9 cell lines. These three cells lines can become resistant to PF-06747775. They represent the first line EGFR-mutant patient population that has progressed on an initial EGFR TKI therapy.

### Example 6

### PF-06747775 and Palbociclib, Alone and in Combination in the H1975 Xenograft Model

Methods:

Four- to six-week-old nude/nude (nu/nu) female mice were obtained from Charles River Laboratories (Hollister, Calif., USA) and maintained in pressurized ventilated caging at the Pfizer La Jolla animal facility. All studies were approved by Pfizer Institutional Animal Care and Use Committees. Xenograft tumors were established by subcutaneously implanting 5×10⁶ H1975 cells suspended 1:1 (v/v) with reconstituted basement membrane (Matrigel, BD Biosciences) in nu/nu mice. For tumor growth inhibition (TGI) studies, mice with established tumors of ˜300-400 mm³ were selected, randomized, and then orally dosed with PF-06747775, palbociclib, or the combination daily at the doses and regimens indicated. Tumor dimensions were measured with vernier calipers and tumor volumes were calculated using the formula of π/6×larger diameter x (smaller diameter)². Tumor growth inhibition percentage (TGI %) was calculated as 100×(1−ΔT/ΔC). Tumor regression percentage was calculated as 100×(1−ΔT/starting tumor size).

PF-06747775 was formulated as a suspension in 0.5% methylcellulose A4M. Palbociclib was formulated as a solution in 50 mM Na lactate buffer. Drugs were formulated once per in vivo experiment and were dosed daily at a concentration of 10 mL/kg through oral gavage. For combination regimen, PF-06747775 was dosed first, then after 5 minutes, palbociclib was dosed.

Results:

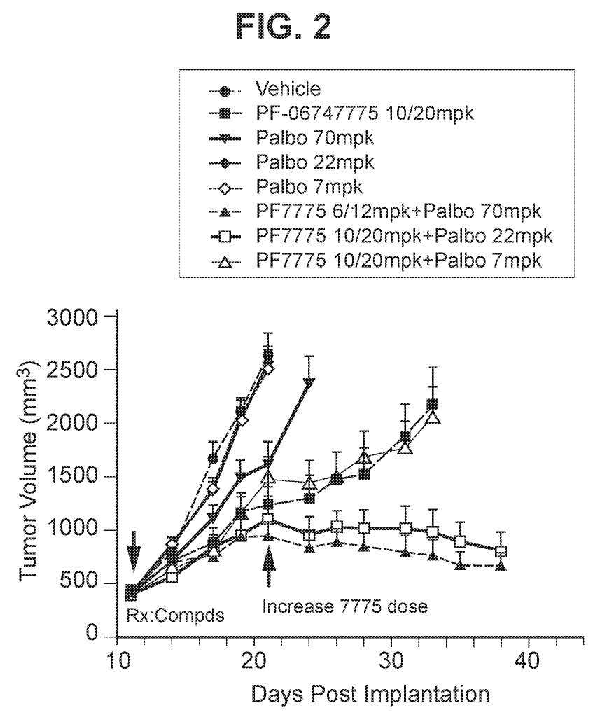

The in vivo anti-tumor efficacy of the combination compared to each single agent was assessed using the H1975 cell line grown as a standard xenograft tumor model in immunocompromised mice (FIG. 2). Palbociclib was dosed at 70 mpk as the clinically relevant dose, and at 22 mpk and 7 mpk to model clinical dose reduction and to identify a dose response. PF-06747775 was initially dosed at 10 mpk (with one exception, see below) to achieve moderate anti-tumor efficacy of the single agent arm to allow for the efficacy from a combination effect to be observed. After 10 days of dosing, the anti-tumor efficacy of PF-06747775 single agent was less than the desired comparator level, hence the PF-06747775 dose was increased to 20 mpk on day 11 in both single agent and combination arms. The exception was the combination arm with 70 mpk palbociclib, where PF-06747775 was initially dosed at 6 mpk, then increased to 12 mpk on day 11. The lower dose of PF-06747775 in this arm was used to offset a previously characterized drug-drug interaction (DDI) observed in combination with palbociclib at 70 mpk that modestly increases the exposure level of PF-06747775. This DDI is due to palbociclib being a mild inhibitor of Cytochrome P-450 3A4, and PF-06747775 being metabolized in vivo by this same enzyme. Blood samples were taken at day 15, and plasma drug levels were determined (Table 5). All arms dosed with PF-06747775 had similar exposure level, indicating the DDI in the 70 mpk palbociclib combination arm was offset by the lower dose of PF-06747775, and the DDI did not occur in combination arms with lower doses of palbociclib. All drug regimens were well tolerated as evidenced by no body weight loss in any treatment arm.

Palbociclib as a single agent had a minor effect on tumor growth inhibition (TGI) at the 70 mpk dose, and no effect at the lower doses (FIG. 2). PF-06747775 as a single agent had a moderate effect on TGI at the 10/20 mpk dose. The combination arm with 7 mpk palbociclib had no additional effect over that of PF-06747775 alone. However the combination arms with 22 mpk and 70 mpk palbociclib showed a clear and substantial increase in TGI over PF-06747775 alone, leading to tumor shrinkage and a trend towards net tumor regression. Hence the combination showed enhanced anti-tumor efficacy in vivo in a tumor model representing the second line T790M resistant patient population.

TABLE 5
Plasma Levels of PF-06747775 and Palbociclib,
Alone and in Combination

| | Plasma Average Free Drug | |
|---|---|---|
| | Concentration Over the 24 hour | |
| | Dosing Interval | |
| | PF-06747775 | Palbociclib |
| Treatment Group | (nM) | (nM) |
| PF-06747775 10/20 mpk | 95 | N/A |
| Palbo 70 mpk | N/A | 127 |
| Palbo 22 mpk | N/A | 45 |
| Palbo 7 mpk | N/A | 6 |
| PF7775 6/12 mpk + Palbo 70 mpk | 119 | 137 |
| PF7775 10/20 + Palbo 22 mpk | 117 | 49 |
| PF7775 10/20 + Palbo 7 mpk | 90 | 6 |

Conclusions:

In vitro and in vivo assessment of the combination of PF-06747775 and palbociclib showed increased efficacy over PF-06747775 alone in NSCLC models representing the first line EGFR-mutant and second line T790M resistant patient populations. These studies represent a potentially clinically translatable strategy to develop EGFR T790M selective inhibitors and CDK inhibitors for increased clinical benefit in EGFR-mutant NSCLC.

1. A method for treating non-small cell lung cancer comprising administering to a patient in need thereof an amount of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and an amount of palbociclib, or a pharmaceutically acceptable salt thereof, wherein the amounts together are effective in treating non-small cell lung cancer.

2. The method of claim 1, wherein the non-small cell lung cancer is EGFR-mutant non-small cell lung cancer.

3. The method of claim 2, wherein the EGFR-mutant non-small cell lung cancer includes a mutation selected from the group consisting of del 19, L858R, del 19/T790M and L858R/T790M.

4. The method of claim 2, wherein the EGFR-mutant non-small cell lung cancer includes a mutation selected from the group consisting of del 19 and L858R.

5. The method of claim 2, wherein the EGFR-mutant non-small cell lung cancer includes a mutation selected from the group consisting of del 19/T790M and L858R/T790M.

6. The method of claim 2, wherein the EGFR-mutant non-small cell lung cancer is advanced EGFR-mutant non-small cell lung cancer.

7. The method of claim 1, wherein palbociclib, or a pharmaceutically acceptable salt thereof, is administered according to a non-standard clinical dosing regimen, and further wherein the amounts together are effective in treating non-small cell lung cancer.

8. The method of claim 7, wherein the non-standard clinical dosing regimen is a non-standard clinical dose.

9. The method of claim 8, wherein the non-standard clinical dose is a low-dose amount of palbociclib, or a pharmaceutically acceptable salt thereof.

10. The method of claim 9, wherein the low-dose amount of palbociclib, or a pharmaceutically acceptable salt thereof, is about 75 mg once daily.

11. The method of claim 7, wherein the non-standard clinical dosing regimen is a non-standard dosing schedule.

12. The method of claim 11, wherein the non-standard dosing schedule is a continuous dosing schedule of palbociclib, or a pharmaceutically acceptable salt thereof.

13. The method of claim 7, wherein the non-standard clinical dosing regimen comprises administering about 75 mg of palbociclib, or a pharmaceutically acceptable salt thereof, once daily for 14 consecutive days followed by 7 days off treatment.

14. A synergistic combination of
(a) N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof; and
(b) palbociclib, or a pharmaceutically acceptable salt thereof,
wherein component (a) and component (b) are synergistic.

15. A combination of N-((3R,4R)-4-fluoro-1-(6-((3-methoxy-1-methyl-1H-pyrazol-4-yl)amino)-9-methyl-9H-purin-2-yl)pyrrolidin-3-yl)acrylamide, or a pharmaceutically acceptable salt thereof, and palbociclib, or a pharmaceutically acceptable salt thereof, for use in the treatment of non-small cell lung cancer.
